(19) United States
(12) Patent Application Publication
(10) Pub. No.: US 2009/0169557 A1
(43) Pub. Date: Jul. 2, 2009

(54) Whole glucan particles in combination with antibiotics, vaccines and viral monoclonal antibodies

(75) Inventors: Gordon D. Ross, Prospect, KY (US); Trunetta Jo Dockter Ross, Prospect, KY (US); Steven J. Karel, Mendota Heights, MN (US); Daniel K. Connors, St.Paul, MN (US); Bill Kournikakis, Medicine Hat (CA)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Biopolymer Engineering, Inc. d/b/a Biothera

(21) Appl. No.: 11/579,467

(22) PCT Filed: May 10, 2005
(86) PCT No.: PCT/US05/16229

§ 371 Date: Oct. 16, 2008

Related U.S. Application Data
(60) Provisional application No. 60/569,559, filed on May 10, 2004.

(51) Int. Cl. A61K 39/395; A61K 31/716; A61K 39/00; A61P 31/12

(52) U.S. Cl. 424/141.1; 514/54; 424/184.1; 424/130.1

(57) ABSTRACT

The present invention relates to compositions and methods of using whole glucan particles and agents. Whole glucan particles enhance the tumoricidal activity of the innate immune system by binding to the C3 complement protein receptor CR3. This binding enhances innate immune system cytotoxicity, as well as stimulating the release of activating cytokines and enhances the bodies response to the agent.

### RELATED APPLICATION

This application claims the benefit of U.S. Provisional Application No. 60/569,559, filed May 10, 2004. The entire teachings of the above application are incorporated herein by reference.

### GOVERNMENT SUPPORT

The invention was supported, in whole or in part, by grant Ro1CA86412 from National Institute for Health/National Cancer Institute and grant BC010287 from the Department of Defense, U.S. Army. The Government has certain rights in the invention.

### BACKGROUND OF THE INVENTION

β-glucan is a complex carbohydrate, generally derived from several sources, including yeast, bacteria, fungi and plants (cereal grains). These sources provide β-glucans in a variety of mixtures, purities and structures. The structural diversity of β-glucan results from the different ways the glucose molecules are able to link yielding compounds with different physical properties and biological properties. For example, β(1,3) glucan derived from bacterial and algae is linear, making it useful as a food thickener. Lentinan (from Lentinus edodes, Basidiomycete family) is a high MW β-glucan with β(1,6) branches off of the (1,3) backbone every three residues.

Schizophyllan (from Schizophyllum commune, Basidiomycetes family) is similar to Lentinan, but with shorter β(1,6) side chains. β-glucan from barley, oat, or wheat has mixed (1,3)- and (1,4)-β-linkages in the backbone, but no (1,6)-β branches, and is generally of high molecular weight. The frequency of side chains, known as the degree of substitution or branching frequency, regulates secondary structure and solubility. β-glucan derived from yeast has a backbone chain of β(1,3) linked glucose units with a low degree of inter and intra-molecular branching through β(1,6) linkages. Based on extensive published research, it is widely accepted that baker's yeast (Saccharomyces cerevisiae) is a preferred source of β(1,3)-glucan, based on the purity and activity of the product obtained.

The cell wall of S. cerevisiae is mainly composed of β-glucans, which are responsible for its shape and mechanical strength. While best known for its use as a food grade organism, yeast is also used as a source of zymosan, a crude insoluble extract used to stimulate a non-specific immune response. Yeast zymosan serves as a rich source of β(1,3) glucan. Yeast-derived beta 1,3 glucan appears to stimulate the immune system, in part, by activating the innate immune system as part of the body's basic defense against fungal infection. Yeast β(1,3) glucan is a polysaccharide composed primarily of β(1,3)-linked glucose molecules with periodic β(1,3) branches linked via β(1,6) linkages and is more formally known as poly-(1,6)-β-D-glucopyranosyl-(1,3)-β-D-glucopyranose. Glucans are structurally and functionally different depending on the source and isolation methods.

β-glucan possess a diverse range of activities. The ability of β-glucan to increase nonspecific immunity and resistance to infection is similar to that of endotoxin. β-glucans' activity as an immune adjuvant and hemopoietic stimulator compares to more complex biological response modifiers such as bacillus Calmette-Guerin and Corynebacterium parvum. The functional activities of yeast β-glucans are also comparable to those structurally similar carbohydrate polymers isolated from fungi and plants. These higher molecular weight (1,3)-β-D-glucans such as schizophyllan, lentinan, krestin, grifolan, and pachyman exhibit similar immunomodulatory activities. Various preparations of both particulate and soluble β-glucans have been tested in animal models to elucidate the biological activities. The use of soluble and insoluble β-glucans alone or as vaccine adjuvants for viral and bacterial antigens has been shown to markedly increase resistance to a variety of bacterial, fungal, protozoan and viral infections. β-glucan's hemopoietic effects include increased peripheral blood leukocyte counts and bone marrow and splenic cellularity, reflecting increased numbers of granulocyte-macrophage progenitor cells, splenic pluripotent stem cells, and erythroid progenitor cells, as well as increased serum levels of granulocyte-monocyte colony-stimulating factor (GM-CSF).

The molecular mechanism of action of β-glucan appears to involve specific β-glucan receptor binding sites on the cell membranes of immune cells such as neutrophils and macrophages. Mannans, galactans, α(1,4)-linked glucose polymers and β(1,4)-linked glucose polymers have no avidity for this receptor. Recent data suggests that CR3, the receptor for C3 complement protein, serves as a major receptor for β-glucans. Ligand binding to the β-glucan receptor results in complement activation, phagocytosis, lysosomal enzyme release, and prostaglandin, thromboxane and leukotriene generation. Most β-glucan preparations described in the prior art stimulate production of cytokines such as IL-1 and TNF, which are known to have anti-tumor activity.

Antibiotics, antivirals and other agents are not always effective alone. In particular, with the growing amount of antibiotic resistance strain, a need exits for developing methods for enhancing the effectiveness of these agents.

### SUMMARY OF THE INVENTION

As described herein, the invention relates to the use of compositions comprising β(1,3;1,6) glucan and an agent, for example an antibiotic, antiviral, antibody, vaccine or combinations thereof.

The invention describes compositions comprising particulate bioavailable β(1,3; 1,6) glucan and an agent, wherein the glucan via the complement system promotes immune responses and the agent activates complement. In certain embodiments, the agent is an antiviral agent such as viral antibody, a vaccine, for example a vaccine for influenza, a anti-rhinovirus agent, and an antibiotic or combinations thereof.

In certain embodiments, the invention pertains to a method of enhancing glucan-mediated immunogenic response via the complement system, comprising administering to an individual a therapeutically effective orally bioavailable amount of whole glucan particles and agent, wherein the agent activates the complement system and the glucan enhances immunogenic response whereby enhancing the activity of the agent.

The orally administered glucan is taken up by macrophages, transported to the bone marrow, degraded and the released fragments activating the immune cells.

The invention further relates to a method of treating or preventing pathogenesis of viral infection in humans or animals by one or more infectious agents comprising administering a prophylactically or therapeutically effective amount of whole glucan particles and an antiviral agent to the human or animal, wherein the glucan activates immune responses and the immune responses enhance the action of the antiviral.

In another aspect, the invention provides a method of treating or preventing pathogenesis of bacterial infection in humans or animals by one or more infectious agents comprising administering a prophylactically or therapeutically effective amount of whole glucan particles and an antibiotic to the human or animal, wherein the agent activates the complement system and the glucan promotes immune responses that enhance the action of the antibiotic.

The invention also pertains to a method of treating or preventing pathogenesis of an infection in humans or animals by one or more infectious agents comprising administering to the human or animal a prophylactically or therapeutically effective amount of whole glucan particles and a vaccine to the infectious agent, wherein the agent activates the complement system and the glucan promotes immune responses whereby the immune responses enhance the action of the antiviral.

In the methods described herein, the glucan can be administered in dosages ranging from about 0 to about 6000 mg per day or about 0 to about 100 mg/kg/day.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a drawing showing the survival data of WGP vs. untreated from Example 1.

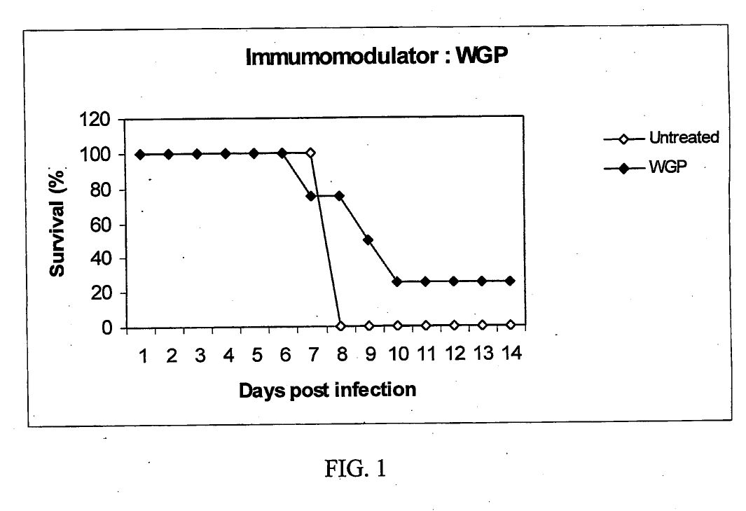

FIG. 2 is a drawing showing the survival data for WGPs, soluble glucan and control, one systemic prophylaxis dose.

FIG. 3 is a drawing showing treatment of for WGPs, soluble glucan and control in a CFU/lung study one systemic prophylaxis dose.

FIG. 4 is a graph showing that effectiveness of WGPs, soluble glucan and control in a bacteria animal model, one systemic prophylaxis dose.

FIG. 5 is a graph showing the survival rate for 2 mg and 20 mg of WGPs, 8 day oral dose.

FIG. 6 is a graph showing the survival rate for 2 mg/kg of WGPs, prophylaxis 4 doses in one week.

FIG. 7 is a graph showing the survival rate for 13.3 mg and 1.5 mg of WGPs 10 days post exposure.

FIG. 8 is a graph showing the influenza protective effect of daily prophylactic oral dosing of WGP β-glucan.

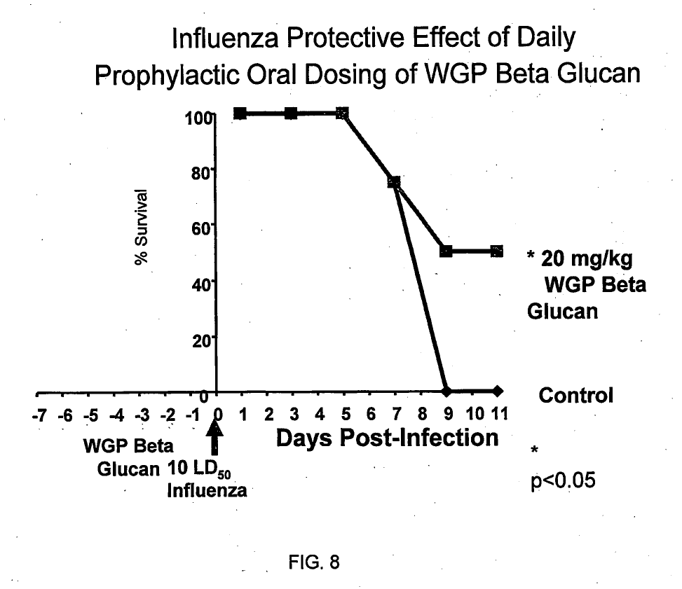

FIG. 9 is a graph showing the survival of ⁶⁰Co γ irradiated B6D2F₁/J Mice following Klebsiella subcutaneous challenge.

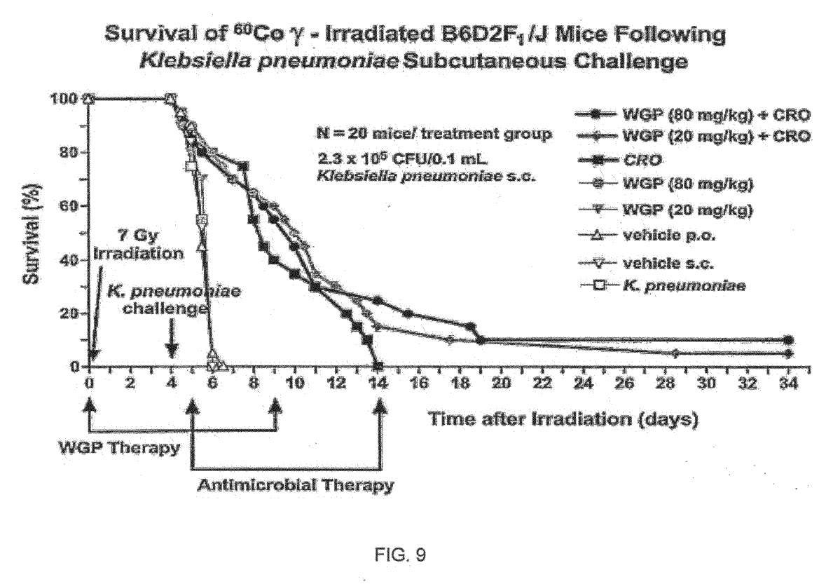

### DETAILED DESCRIPTION OF THE INVENTION

A description of preferred embodiments of the invention follows.

Whole glucan particles (WGPs) comprise a preparation of purified, insoluble yeast cell walls. WGPs are produced by removing the mannan protein outer layer and exposing the β-glucan while retaining glucan's in vivo morphology.

Whole glucan particles are the remnants of the yeast cell wall prepared by separating growing yeast from its growth medium and subjecting the intact cell walls of the yeast to alkali, thus removing unwanted proteins and nucleic acid material. In certain embodiments, what remains is a spherical β-glucan particle with the outer mannan protein removed. Whole glucan particles may be obtained from any glucan-containing cell wall source, but the preferred source is a strain of S. cerevisiae. In certain embodiments, the glucan content of preparations is greater than 50% glucan. The remainder can be comprised of intracellular lipids and/or glycogen. These insoluble particles have been shown to enhance host resistance to a wide range of infections, increase antibody production (adjuvant activity), increase leukocyte mobilization, and enhance wound healing. Methods of producing WGP are known in the art and are disclosed in U.S. Pat. Nos. 4,810,646, 4,492,540, 5,037,972, 5,082,936, 5,250,436, and 5,506,124, the contents of which are incorporated herein by reference in their entirety.

Microparticulate glucan particles are defined herein to be portions of whole glucan particles that result from finely grinding yeast cell wall β(1,3; 1,6) glucan down to a particle size of about 1 micron or less. Microparticulate β-glucan have also been shown to enhance the host's immune system. See U.S. Pat. Nos. 5,223,491 and 5,576,015, the teachings of which are incorporated herein by reference in their entirety.

Various forms of particulate β-glucans have been prepared. One example is microparticulate whole glucan particles, which can be formed by finely grinding yeast cell wall β(1,3;1,6) glucan down to a particle size of about 1 micron or less. β-glucan in this form has been described for use as a nutritional supplement and skin restorer, such as disclosed in U.S. Pat. No. 5,702,719, by Donzis. Other particulate glucans useful in the methods described herein, are WGP® Beta Glucan and BetaRight™ obtained from Biopolymer Engineering, Inc., Eagan, Minn.

Microparticulate β-glucan have also been shown to enhance the host's immune system. See U.S. Pat. Nos. 5,223,491 and 5,576,015, the teachings of which are incorporated herein by reference in their entirety.

Described herein, are compositions comprising β(1,3;1,6) glucan and an agent, wherein the β(1,3;1,6) glucan stimulates the immune system and improves the agents ability to act by priming the body. In certain embodiments, whole glucan particles for use in the compositions in the methods described herein are oral bioavailable formulations. “Bioavailable”, as used herein, means the whole glucan particle is able to reach the target of action. In other words, whole glucan particles when administered provide enough β(1,3; 1,6) glucan exposed for Peyer's patch uptake of the glucan. The glucan is taken up in the Peyer's patch, engulfed and degraded by macrophages, and then transported to the bone marrow where the degraded fragments are released. The released degraded fragments bind to neutrophils in the bone marrow and through chemotaxis migrate to and bind to the target site where complement has been activated via deposited iC3b. For example, the WGP in the compositions described herein is able to reach and act on target cells in combination with the agent and enhance the agent's activity.

At the site of action, the glucan acts to stimulate cells as a result of the binding or association of the glucan to the CR3 receptor. The binding results in the priming and/or promotion of CR3. The bioavailability of oral WGP is mediated by the transport of WGP to the bone marrow by gastrointestinal macrophages that degrade the glucan particle. The degraded particles then function at the bone marrow as stimulators of neutrophils via CR3 activation when the neutrophils migrate to cells and bind to the cells with deposited iC3b. In conjunction with the activity of the agent, the activation of the immune system via glucans enables the body to better respond to the agent.

β-Glucan, a well-known biological response modifier (BRM), stimulates hematopoiesis (blood cell formation), in an analogous manner as granulocyte monocyte-colony stimulating factor (GM-CSF). Research was carried out initially with particulate β-glucan and later with soluble β-glucans, all of which were administered intravenously to mice (Patchen M. L., et al., J. Biol. Response Mod. 3:627-633 (1984), Patchen, M. L., et al., Experientia 40:1240-1244 (1984), Petruczenko, A. Acta. Physiol. Pol. 35:231-236 (1984) and Patchen, M. L. and T. J. MacVittie., Int. J. Immunopharmacol. 7:923-932 (1985)). Mice exposed to 500-900 cGy of gamma radiation exhibited a significantly enhanced recovery of blood leukocyte, platelet and red blood cell counts when given i.v. β-glucans (Patchen, M. L. and T. J. MacVittie. J. Biol. Response Mod. 5:45-60 1986) and Patchen, M. L., et al., Methods Find. Exp. Clin. Pharmacol. 8:151-155 (1986)). Other reports showed that β-glucan could reverse the myelosuppression produced with chemotherapeutic drugs such as fluorouracil (Matsuo, T., et al., Jpn. J. Cancer Chemother. 14: 1310-1314 (1987) or cyclophosphamide (Wagnerová, J., et al., Immunopharmacol. Immunotoxicol. 15:227-242 (1993) and Patchen, M. L et al., Exp. Hematol. 26:1247-1254 (1998)). Moreover, the anti-infective activity of β-glucan combined with its hematopoiesis-stimulating activity resulted in enhanced survival of mice receiving a lethal dose of 900-1200 cGy of radiation. In vitro studies showed that β-glucan could enhance granulocyte and megakaryocyte colony formation by hematopoietic stem progenitor cells when used in combination with GM-CSF and interleukin-3 (IL-3), respectively (Turnbull, J. L et al., Acta Haematol. 102:66-71 (1999)). Development of β-glucans for their hematopoietic activity was not considered worthwhile at that time because of advent of GM-CSF as a therapeutic agent. The Armed Forces Radiobiological Research Institute (AFRRI) that did much of the early research showing the radioprotective effects of β-glucan also considered β-glucan use to protect individuals exposed to radiation as a result of a nuclear power plant accident or nuclear war. However, the apparent need to administer β-glucans intravenously made it unfeasible to rapidly treat large numbers of people in such emergency situations.

As described herein, the oral immunomodulatory activities of β-glucans and benefits associated with the co-administration with various agents, for example vaccines, have been recognized. It is believed that the oral uptake of certain β-glucans by M (microfold) cells in intestinal Peyer's patches leads to β-glucan presentation to macrophages in the underlying gut-associated lymphatic tissue (GALT), the activation of the immune system in this manner results in the body having a increased response to the co-administered agent.

Orally delivered mushroom β-glucans have been shown to activate peritoneal and alveolar macrophages. Further, oral administration of the shitake mushroom-derived β-glucan, lentinan has been found to increase the number of T helper cells in blood of rats. Oral β-glucan has also been shown to induce anti-infective (Hotta, H., K. et al., Int. J. Immunopharmacol. 15:55-60 (1993) and Vetvicka, V. K. J. Amer. Nutrit. Assoc. 5:1-5 (2002)) and anti-tumor activities in both preclinical and clinical studies (Nanba, H. K. et al., Chem. Pharm. Bull. (Tokyo) 35:2453-2458 (1987); Suzuki, I. T. et al., Chem. Pharm. Bull. (Tokyo) 39:1606-1608 (1991) and Toi, M. T. et al., Cancer 70:2475-2483 (1992)). Without being bound by theory, β-glucans function by stimulating host immune defense mechanisms, primarily macrophages, neutrophils, NK cells, and dendritic cells, thereby enhancing microbial or tumor cell clearance and subsequently reducing mortality (Onderdonk, A., et al., Infect. Immun. 60:1642-1647 (1992) and Kaiser, A. B. and D. S. Kernodle., Antimicrob. Agents Chemother. 42:2449-2451 (1998)).

Yeast-derived β(1,3; 1,6) glucans work, in part, by stimulating innate anti-fungal immune mechanisms to fight a range of pathogenic challenges from bacteria, fungi, parasites, viruses, and cancer. Research to define the mechanism of action of β-glucans has shown that they function through the priming of macrophages, neutrophils, monocytes, and NK cells, giving these cells an enhanced activity to kill microbial pathogens or tumor cells. β-glucans from various sources with different structures have been shown to bind to a variety of receptors. Mannans, galactans, α(1,4)-linked glucose polymers and β(1,4)-linked glucose polymers have no avidity for the receptor located on the cells. Two β-glucan-binding receptors on leukocytes have been characterized that function to promote the phagocytosis of yeast cells walls via binding to β-glucan. First, the iC3b-receptor CR3 (also known as Mac-1, CD11b/CD18, or α_{M}β₂-integrin) was shown to have a β-glucan-binding lectin site that functioned in the phagocytosis of yeast cell walls by neutrophils, monocytes, and macrophages (Ross, G. D., et al., Complement Inflamm. 4:61-74 (1987) and Xia, Y. V. et al., J. Immunol. 162:2281-2290 (1999)). Mac-1/CR3 functions as both an adhesion molecule mediating the diapedesis of leukocytes across the endothelium and a receptor for the iC3b fragment of complement responsible for phagocytic/degranulation responses to microorganisms. Mac-1/CR3 has many functional characteristics shared with other integrins, including bidirectional signaling via conformational changes that originate in either the cytoplasmic domain or extracellular region. Another key to its functions is its ability to form membrane complexes with glycosylphosphatidylinositol (GPI)-anchored receptors such as Fc gammaRIIIB (CD16b) or uPAR (CD 87), providing a transmembrane signaling mechanism for these outer membrane bound receptors that allows them to mediate cytoskeleton-dependent adhesion or phagocytosis and degranulation. Many functions appear to depend upon a membrane-proximal lectin site responsible for recognition of either microbial surface polysaccharides or GPI-linked signaling partners. Because of the importance of Mac-1/CR3 in promoting neutrophil inflammatory responses, therapeutic strategies to antagonize its functions have shown promise in treating both autoimmune diseases and ischemia/reperfusion injury. Conversely, soluble β-glucan polysaccharides that bind to its lectin site prime the Mac-1/CR3 of circulating phagocytes and natural killer (NK) cells, permitting cytotoxic degranulation in response to iC3b-opsonized tumor cells that otherwise escape from this mechanism of cell-mediated cytotoxicity. CR3 binds soluble fungal β-glucan with high affinity (5×10⁻⁸M) and this primes the receptor of phagocytes or NK cells for cytotoxic degranulation in response to iC3b-coated tumor cells. The tumoricidal response promoted by soluble β-glucan in mice was shown to be absent in mice deficient in either serum C3 (complement 3) or leukocyte CR3, highlighting the requirement for iC3b on tumors and CR3 on leukocytes in the tumoricidal function of β-glucans Vetvicka, V., et al., J. Clin. Invest. 98:50-61 (1996) and Yan, J. V. et al., J. Immunol. 163:3045-3052 (1999)).

Dectin-1 represents the second membrane receptor for β-glucan involved with glucan particle phagocytosis. Dectin-1 is expressed at high levels on thioglycolate-elicited peritoneal macrophages and its activity predominates over that of CR3 in the phagocytosis of yeast via β-glucan binding by these activated cells. However, yeast phagocytosis by neutrophils and resident peritoneal macrophages is blocked by anti-CR3 and does not occur with CR3-deficient (CD11b^{-/}) neutrophils or resident macrophages. Moreover, dectin-1 is not expressed by NK cells that use CR3 to mediate tumoricidal activity against iC3b-opsonized mammary carcinoma cells following priming with β-glucan. Thus the role of dectin-1 in mediating β-glucan activities appears to be limited to activated peritoneal macrophages and perhaps also the intestinal CR3^{-/-} macrophages observed to contain WGP-DTAF in this investigation.

### Immunostimulatory Properties

The methods and compositions described herein, utilize whole glucan particles in combination with an agent (e.g., viral antibody, vaccine, antibiotic) to augment, stimulate, activate, potentiate, or modulate the immune response at either the cellular or humoral level. The mode of action of using the compositions and methods described herein may be either non-specific, e.g., resulting in increased immune responsiveness to a wide variety of antigens, or antigen-specific, e.g., affecting a restricted type of immune response to a narrow group of cell and/or antigens.

The compositions and methods of the present invention can facilitate not only the activation and proliferation of immune cells but also their recruitment. Thus, administration of the compositions of the instant invention facilitate the migration of immune cells using the interaction of the WGP with complement into a specific area, for example, a tumor where the immune cells may become activated and proliferate and the delivery of an agent to the target (e.g., tumor) where the agent then acts of the target site. While activation and proliferation in response to the properties of the present invention can be non-specific, the presence of the agent, (e.g., antibody, vaccine, antibiotic) in the vicinity of the immune cells facilitates the progression to an antigen specific response.

The compositions of the present invention contain agents that provide a benefit to the individual while taking advantage of the glucan's immunomodulatory properties. In certain embodiments, the compositions provide the benefits of the agent but also through the action of the delivery of the glucan and the activation of the complement cascade facilitate the presentation and delivery of these relevant agents to immune cells within a single preparation.

The agents can be incorporated within or commingled with the glucan particles, covalently linked, or provided in a suspension, emulsion or other medium.

In certain embodiments, a benefit of the disclosed system and coadministration is that the preparation may be engineered to further comprise the agents in a single composition. Thus, the benefits afforded by use of the whole glucan particle preparation can be further enhanced by the inclusion of an agent. Therefore, in one embodiment of the invention, the preparation is a glucan with the agent incorporated. For example, compositions contemplated are a whole glucan particles with a viral antibody incorporated within.

A preparation can then be directly introduced into the target site, thus leading not only to the recruitment and potential activation of the immune cells by the glucan binding to the lectin site and activating complement, but the preparation also possesses the further benefit accorded by the inclusion of the agent in the preparation. For example, a vaccine may be combined with the glucan, delivered to the macrophage engulfed and presented to initiate antigen-specific and glucan modulated immune responses.

One role of the instant invention is in the induction of an effective protective immune response. Additionally, a significant component of the claimed compositions is the ability of the composition to preferentially activate and induce the proliferation and or recruitment of immune cells and with the presence of the agent, an immune response to a specific target. The adjuvant properties of the compositions, described herein, facilitate a specific immunological response. Further, it is envisioned that the compositions of the instant invention can further comprise both antigenic components and/or immunomodulators. The combination with an antigenic agent will further facilitate the establishment of the desired immunological response and allow for the creation of immunological memory.

Agents Included with the β-Glucan Compositions

### Antigens/Antibodies

In one aspect, the invention provides an agent comprising an antigenic or immunogenic epitope. Compounds or molecules comprising an immunogenic epitope are those agents capable of inducing an immune response. An “immunogenic epitope” is defined as a part of an agent that elicits an immune response when the whole agent is the immunogen. These immunogenic epitopes are generally confined to a few loci on the molecule. For the purposes of the instant invention, the term “immunogen” or “immunogenic epitope” is not confined to the induction of solely a humoral or solely a cellular response. Rather, the term is used to denote the capability of a compound, molecule or agent to induce either or both a cellular and a humoral immune response.

As to the selection agents bearing an immunogenic epitope it is well known in that art that specific conformations preferentially lead to the induction of a specific form of immune response. For example, peptides capable of eliciting protein-reactive sera as frequently represented in the primary sequence of a protein, can be characterized by a set of simple chemical rules, and are confined neither to immunodominant regions of intact proteins (i.e., immunogenic epitopes) nor to the amino or carboxyl terminals. For example the antibody can be directed to the protein coat of a virus.

### Antibiotics

In certain embodiments, the compositions comprise β(1,3;1,6) glucan and an antibiotic. Antibiotics, also known as antimicrobial drugs, are drugs that fight infections caused by bacteria. After their discovery in the 1940's they transformed medical care and dramatically reduced illness and death from infectious diseases. However, over the decades the bacteria that antibiotics control have developed resistance to these drugs. Today, virtually all important bacterial infections in the United States and throughout the world are becoming resistant. For this reason, antibiotic resistance is among CDC's top concerns.

Antibiotics include penicillins, cephalosporins, macrolides (Erythromycin and its Relatives), sulfanilamides or sulfonamides, Trimethoprim-Sulfamethoxazole, Nitrofurantoin, Aminoglycosides and Polymyxin B among others. A specific antibiotic for use as an agent in the present invention is ciprofloxacin.

The vast majority of antibiotics are either penicillins or cephalosporins; chemical changes have been made to the molecules over the years to improve their bacteria-fighting abilities and to help them overcome breakdown and “immunity” of resistant bacteria.

### Vaccines

In certain embodiments, the compositions comprise β(1,3;1,6) glucan and a vaccine. Vaccines have been developed for the prevention of several significant human diseases of viral origin. Viral vaccines can generally be divided into the following three groups: (i) live, attenuated, (ii) inactivated, and (iii) subunit, based on the nature of the active agents of the vaccines. Each of these types of vaccines has its own distinct advantages and manners of production. Live, attenuated vaccines, for example, simulate natural infections and thus stimulate long-lasting antibody production, induce a good cell-mediated response, and induce resistance at the point of entry. These vaccines have generally been produced in primary cell lines, chick embryos, and diploid cell lines. Inactivated or killed vaccines, in contrast, typically stimulate only a brief immune response, and thus require periodic boosting.

### Flu Vaccine

In certain embodiments, the compositions comprise β(1,3;1,6) glucan and a flu vaccine. The flu vaccine given parenterally (injected as a shot) is inactivated (killed) influenza vaccine. The nasal-flu vaccine is live attenuated influenza that contains weakened virus in contrast to the killed virus used with the shot. When the viruses are sprayed into the nose, they stimulate the body's immune system to develop protective antibodies that will prevent infection by the naturally occurring influenza virus. These viruses are attenuated meaning they are cold-adapted and temperature sensitive meaning they can grow in the nose and throat, but not in the lower respiratory tract where the temperature is higher.

Antivirals and Antiviral with the Flu Vaccine

In certain embodiments, the compositions comprise β(1,3;1,6) glucan and an antiviral agent. In another embodiment, the compositions comprise β(1,3; 1,6) glucan a flu vaccine and an antiviral. According to the Center of Disease Control (CDC), four antiviral drugs are currently approved and commercially available (amantadine, rimantadine, zanamavir and oseltamivir). These drugs are about 70% to 90% effective for preventing illness against influenza viruses in healthy adults.

If taken within 2 days of getting sick, these drugs can reduce the symptoms of the flu and shorten the time one is sick by 1 or 2 days. The antiviral drugs also make one less contagious to others.

The use of these antiviral drugs is often in the control of flu outbreaks in institutions (e.g., nursing homes, hospitals) or on cruise ships or other settings to control outbreaks of the flu. In the event of an outbreak, public health practice is to use the flu vaccine and antivirals.

In certain embodiments, the compositions comprise β(1,3;1,6) glucan and a antiflavivirus. Flaviviruses are members of a family of small, enveloped positive-strand RNA viruses, some of the members of which pose current or potential threats to global public health. For example, Japanese encephalitis is a significant public health problem involving millions of at risk individuals in the Far East. Dengue virus, with an estimated annual incidence of 100 million cases of primary dengue fever and over 450,000 cases of dengue hemorrhagic fever worldwide, has emerged as the single most important arthropod-transmitted human disease.

Other flaviviruses continue to cause endemic diseases of variable nature and have the potential to emerge into new areas as a result of changes in climate, vector populations, and environmental disturbances caused by human activity. These flaviviruses include, for example, St. Louis encephalitis virus, which causes sporadic, but serious, acute disease in the midwest, southeast, and western United States; West Nile virus, which causes febrile illness, occasionally complicated by acute encephalitis, and is widely distributed throughout Africa, the Middle East, the former Soviet Union, and parts of Europe; Murray Valley encephalitis virus, which causes endemic nervous system disease in Australia; and Tick-borne encephalitis virus, which is distributed throughout the former Soviet Union and eastern Europe, where its Ixodes tick vector is prevalent and responsible for a serious form of encephalitis in those regions.

Hepatitis C virus (HCV) is another member of the flavivirus family, with a genome organization and a replication strategy that are similar, but not identical, to those of the flaviviruses mentioned above. HCV is transmitted mostly by parenteral exposure and congenital infection, is associated with chronic hepatitis that can progress to cirrhosis and hepatocellular carcinoma, and is a leading cause of liver disease requiring orthotopic transplantation in the United States.

### Rhinovirus

In certain embodiments, the compositions comprise β(1,3;1,6) glucan and a antirhinovirus agent. Rhinovirus is the most frequent cause of the common cold responsible for 30-50% of cases. A cold is an acute infection of the upper respiratory tract; characterized by coryza, sneezing, lacrimation, irritated nasopharynx, headache, sore throat, chilliness and malaise lasting 2-7 days; little or no fever; can be accompanied by laryngitis, tracheitis and bronchitis; secondary bacterial infection may produce acute otitis media, sinusitis or pneumonitis

Rhinoviruses (RVs) are small (30 nm), nonenveloped viruses that contain a single-strand ribonucleic acid (RNA) genome within an icosahedral (20-sided) capsid. RVs belong to the Picornaviridae family, which includes the genera Enterovirus (polioviruses, coxsackieviruses groups A and B, echoviruses, numbered enteroviruses) and Hepatovirus (hepatitis A virus). Approximately 101 serotypes are identified currently.

The common cold is most frequently is associated with rhinovirus (RV). Nasopharyngitis, croup, and pneumonia are in some cases caused by RV. RV additionally plays a significant role in the pathogenesis of otitis media and asthma exacerbations.

RV can be transmitted by aerosol or direct contact. Primary site of inoculation is the nasal mucosa, although the conjunctiva may be involved to a lesser extent. RV attaches to respiratory epithelium and spreads locally. The major human RV receptor is intercellular adhesion molecule-1 (ICAM-1). The natural response of the human defense system to injury involves ICAM-1, which aids the binding between endothelial cells and leukocytes. RV takes advantage of the ICAM-1 by using it as a receptor for attachment. In addition, RV uses ICAM-1 for subsequent viral uncoating during cell invasion. Some RV serotypes also up-regulate the ICAM-1 expression on human epithelial cells to increase infection susceptibility.

Optimum environment for RV replication is 33-35° C. RV does not replicate efficiently at body temperature. This may explain why RV replicates well in the nasal passages and upper tracheobronchial tree but less well in the lower respiratory tract. Incubation period is approximately 2-3 days.

RV is shed in large amounts, with as many as 1 million infectious virions per milliliter of nasal washings. Viral shedding can occur a few days before cold symptoms are recognized by the patient, peaks on days 2-7 of the illness, and may last for as many as 3-4 weeks.

A local inflammatory response to the virus in the respiratory tract can lead to nasal discharge, nasal congestion, sneezing, and throat irritation. Damage to the nasal epithelium does not occur, and inflammation is mediated by the production of cytokines and other mediators.

Histamine concentrations in nasal secretions do not increase. By days 3-5 of the illness, nasal discharge can become mucopurulent from polymorphonuclear leukocytes that have migrated to the infection site in response to chemoattractants such as interleukin-8. Nasal mucociliary transport is reduced markedly during the illness and may be impaired for weeks. Both secretory immunoglobulin A and serum antibodies are involved in resolving the illness and protecting from reinfection.

Coronaviruses, reinfections with parainfluenza, and respiratory syncytial virus (RSV) are the most important of many other viruses that can cause common colds. Other viruses (e.g., adenoviruses, influenza viruses) also can cause common colds but are more likely to cause acute nasopharyngitis and more severe respiratory infections.

Mycoplasma pneumoniae occasionally can present with common cold symptoms before developing into more extensive respiratory disease. Other pathogens include Coccidioides immitis, Histoplasma capsulatum, Bordetella pertussis, Chlamydia psittaci, and Coxiella burnetii.

Recent clinical studies indicate sinus involvement in common colds. CT scan abnormalities (e.g., opacification, air-fluid levels, mucosal thickening) are present in adults with common colds that resolve over 1-2 weeks without antibiotic therapy.

In certain embodiments, the compositions comprise β(1,3;1,6) glucan and a Pleconaril. Pleconaril is a viral capsid-binding inhibitor with potent and highly specific in vitro activity against the majority of serotypes of rhinoviruses and enteroviruses.

### Preparation of WGP Glucan

Briefly, the process for producing whole glucan particles involves the extraction and purification of the alkali-insoluble whole glucan particles from the yeast or fungal cell walls. This process yields a product, which maintains the morphological and structural properties of the glucan as, found in vivo.

The structure-function properties of the whole glucan preparation depend directly on the source from which it is obtained and also from the purity of the final product. The source of whole glucan can be yeast or other fungi, or any other source containing glucan having the properties described herein. In certain embodiments, yeast cells are a preferred source of glucans. The yeast strains employed in the present process can be any strain of yeast, including, for example, S. cerevisiae, S. delbrueckii, S. rosei, S. microellipsodes, S. carlsbergensis, S. bisporus, S. fermentati, S. rouxii, Schizosaccharomyces pombe, Kluyveromyces polysporus, Candida albicans, C. cloacae, C. tropicalis, C. utilis, Hansenula wingei, H. arni, H. henricii, H. americana, H. canadiensis, H. capsulata, H. polymorpha, Pichia kluyveri, P. pastoris, P. polymorpha, P. rhodanensis, P ohmeri, Torulopsis bovin and T. glabrata.

Generally, the above procedure can be used to prepare and isolate other mutant yeast strains with other parent strains as starting material. Additionally, mutagens can be employed to induce the mutations, for example, chemical mutagens, irradiation, or other DNA and recombinant manipulations. Other selection or screening techniques may be similarly employed.

The yeast cells may be produced by methods known in the art. Typical growth media comprise, for example, glucose, peptone and yeast extract. The yeast cells may be harvested and separated from the growth medium by methods typically applied to separate the biomass from the liquid medium. Such methods typically employ a solid-liquid separation process such as filtration or centrifugation. In the present process, the cells are preferably harvested in the mid-to late logarithmic phase of growth, to minimize the amount of glycogen and chitin in the yeast cells. Glycogen, chitin and protein are undesirable contaminants that affect the biological and hydrodynamic properties of the whole glucan particles.

Preparation of whole glucan particles involves treating the yeast with an aqueous alkaline solution at a suitable concentration to solubilize a portion of the yeast and form an alkali-hydroxide insoluble whole glucan particles having primarily β(1,6) and β(1,3) linkages. The alkali generally employed is an alkali-metal hydroxide, such as sodium or potassium hydroxide or an equivalent. The starting material can comprise yeast separated from the growth medium. It is more difficult to control consumption of the aqueous hydroxide reactants and the concentration of reactants in the preferred ranges when starting with yeast compositions that are less concentrated. The yeast should have intact, unruptured cell walls since the preferred properties of the instant whole glucan particles depend upon an intact cell wall.

The yeast are treated in the aqueous hydroxide solution. The intracellular components and mannoprotein portion of the yeast cells are solubilized in the aqueous hydroxide solution, leaving insoluble cell wall material which is substantially devoid of protein and having a substantially unaltered three dimensional matrix of β(1,6) and β(1,3) linked glucan. The preferred conditions of performing this step result in the mannan component of the cell wall being dissolved in the aqueous hydroxide solution. The intracellular constituents are hydrolyzed and released into the soluble phase. The conditions of digestion are such that at least in a major portion of the cells, the three dimensional matrix structure of the cell walls is not destroyed. In particular circumstances, substantially all the cell wall glucan remains unaltered and intact.

In certain embodiments, the aqueous hydroxide digestion step is carried out in a hydroxide solution having initial normality of from about 0.1 to about 10.0. Typical hydroxide solutions include hydroxides of the alkali metal group and alkaline earth metals of the Periodic Table. The preferred aqueous hydroxide solutions are of sodium and potassium, due to their availability. The digestion can be carried out at a temperature of from about 20° C. to about 121° C. with lower temperatures requiring longer digestion times. When sodium hydroxide is used as the aqueous hydroxide, the temperature can be from about 80° C. to about 100° C. and the solution has an initial normality of from about 0.75 to about 1.5. The hydroxide added is in excess of the amount required, thus, no subsequent additions are necessary.

Generally from about 10 to about 500 grams of dry yeast per liter of hydroxide solution is used. In certain embodiments, the aqueous hydroxide digestion step is carried out by a series of contacting steps so that the amount of residual contaminants such as proteins are less than if only one contacting step is utilized. In certain embodiments, it is desirable to remove substantially all of the protein material from the cell. Such removal is carried out to such an extent that less than one percent of the protein remains with the insoluble cell wall glucan particles. Additional extraction steps are preferably carried out in a mild acid solution having a pH of from about 2.0 to about 6.0. Typical mild acid solutions include hydrochloric acid, sodium chloride adjusted to the required pH with hydrochloric acid and acetate buffers. Other typical mild acid solutions are in sulfuric acid and acetic acid in a suitable buffer. This extraction step is preferably carried out at a temperature of from about 20° C. to about 100° C. The digested glucan particles can be, if necessary or desired, subjected to further washings and extraction to reduce the protein and contaminant levels. After processing the product pH can be adjusted to a range of about 6.0 to about 7.8.

By conducting this process without a step of disrupting the cell walls, the extraction can be conducted at more severe conditions of pH and temperature than was possible with the prior art procedure that included a step of disrupting the cell walls. That is, the process of this invention avoids product degradation while employing these severe extraction conditions which permits elimination of time-consuming multiple extraction steps.

After the above aqueous hydroxide treatment step, the final whole glucan product comprises about 5 to about 30 percent of the initial weight of the yeast cell, preferably the product is from about 7 to about 15 percent by weight. The aqueous hydroxide insoluble whole glucan particles produced is as set forth in the summary of the invention. The whole glucan particles can be further processed and/or further purified, as desired. For example, the glucan can be dried to a fine powder (e.g., by drying in an oven); or can be treated with organic solvents (e.g., alcohols, ether, acetone, methyl ethyl ketone, chloroform) to remove any traces or organic-soluble material, or retreated with hydroxide solution, to remove additional proteins or other impurities that may be present.

In certain embodiments, the whole glucan particles obtained from the present process are comprised of pure glucan, which consists essentially of β(1,6) and β(1,3) linked glucan. The whole glucan particles contain very little contamination from protein and glycogen. In certain embodiments, the whole glucan particles are spherical in shape with a diameter of about 2 to about 10 microns and contain greater than about 85% by weight hexose sugars, (or in other embodiments greater than about 60% hexose sugars), approximately 1% by weight protein and less than 1% of a detectable amount of mannan, as determined monosaccharide analysis or other appropriate analysis. Glucans obtained by prior processes contain substantially higher quantities of chitin and glycogen than the present glucans.

The second step as set forth above, involves the modification of the whole glucan particles, as produced above, by chemical treatment to change the properties of the glucan. It is contemplated that whole glucan particles derived from any yeast strain may be used, in addition to those particular strains described herein, As mentioned above, a very broad spectrum of yeast or mutant yeast strains may be used. The processing conditions described above are also applicable to glucan extraction from fungi in general. The properties of these glucans also will depend on the sources from which they are derived.

According to a first chemical treatment, the whole glucan particles can be treated with an acid to decrease the amount of β(1,6) linkages and thus, change the hydrodynamic properties of said glucans as evidenced by an increase in the viscosity of aqueous solutions of these modified glucans.

A process for preparing an altered whole glucan particles by treating the glucan particles with an acid, for a suitable period of time to alter the β(1,6) linkages can also be used. Acetic acid is preferred, due to its mild acidity, ease of handling, low toxicity, low cost and availability, but other acids may be used. Generally these acids should be mild enough to limit hydrolysis of the β(1,3) linkages. The treatment is carried out under conditions to substantially only affect the β(1,6) linked glucans. In certain embodiments, the acid treatment is carried out with a liquid consisting essentially of acetic acid, or any dilutions thereof (typical diluents can be organic solvents or inorganic acid solutions). The treatment is carried out at a temperature of from about 20° C. to about 100° C. In certain embodiments, the treatment is carried out to such an extent to remove from about 3 to about 20 percent by weight of acid soluble material based on total weight of the whole glucan particles before treatment. In other embodiments, the extent of removal is from about 3 to about 4 percent by weight. Certain compositions formed demonstrate altered hydrodynamic properties and an enhancement in viscosity after treatment.

According to a second chemical treatment, the whole glucan particles are treated with an enzyme or an acid, to change the amount of β(1,3) linkages. For whole glucan particles derived from some yeast strains, enzyme treatment causes a decrease in the viscosity, and for others, it causes an increase in viscosity, but in general, alters the chemical and hydrodynamic properties of the resulting glucans. The treatment is with a β(1,3) glucanase enzyme, such as laminarinase, for altering the β(1,3) linkages to alter the hydrodynamic properties of the whole glucan particles in aqueous suspensions.

The enzyme treatment can be carried out in an aqueous solution having a concentration of glucan of from about 0.1 to about 10.0 grams per liter. Any hydrolytic glucanase enzyme can be used, such as laminarinase, which is effective and readily available. The time of incubation may vary depending on the concentration of whole glucan particles and glucanase enzyme. The β(1,3) linkages are resistant to hydrolysis by mild acids such as acetic acid. Treatment with strong or concentrated acids, such as hydrochloric acid (HCl ), sulfuric acid (H₂SO₄) or formic acid, hydrolyzes the β(1,3) linkages thereby reducing the amount of β(1,3) linkages. The acid treatment can be carried out in an aqueous solution having a concentration of glucan from about 0.1 to about 10.0 grams per liter. The time of acid treatment may vary depending upon the concentration of whole glucan particles and acid. Acid hydrolysis can be carried out at a temperature of from about 20° C. to about 100° C. The preferred compositions formed demonstrate altered hydrodynamic properties.

By controlling the incubation time, it is possible to control the chemical and hydrodynamic properties of the resulting product. For example, the product viscosity can be precisely controlled for particular usage, as, for example, with a variety of food products.

A hydrodynamic parameter (K₁) of the final treated product having altered linkages is dependent on the treatment time according to the final formula:

K₁=−0.0021(time)+0.26

where time is in minutes; and
where time is less than one hour.

The parameter K₁ is directly related (proportional) to the relative viscosity. In the case of aqueous suspensions the relative viscosity is equal to the actual viscosity when the latter is measured in centipoise.

A process for preparing aqueous slurry of a glucan having a predetermined desired viscosity is provided. The slurry comprises glucan at a concentration that is a function of the predetermined desired viscosity according to the following approximate formula:

1/concentration=K₁×(1/log(relative viscosity))+K₂

### Where,

K₁=(shape factor)×(hydrodynamic volume); and
K₂=(hydrodynamic volume)/(maximum packing fraction).

The shape factor is an empirically determined value that describes the shape of the glucan matrix in its aqueous environment. The shape factor is a function of the length:width ratio of a particle and can be determined microscopically. The hydrodynamic volume is a measure of the volume a particle occupies when in suspension. This is an important parameter for glucan suspensions in that it indicates the high water holding capacity of glucan matrices. The maximum packing fraction can be described as the highest attainable volume fraction of glucans that can be packed into a unit volume of suspension.

Preparation of Microparticulate β-Glucan Particles β(1,3) glucan starting material can be isolated from yeast cell walls by conventional methods known by those of ordinary skill in the art. The general method for the production of glucan from yeast involves extraction with alkali followed by extraction with acid (Hassid et al., Journal of the American Chemical Society, 63:295-298, 1941). Improved methods for isolating a purified water insoluble β(1,3) glucan extract are disclosed in U.S. Pat. No. 5,223,491, which is incorporated herein by reference in its entirety. Another method of producing whole glucan particles is disclosed in U.S. Pat. No. 4,992,540, which is incorporated herein by reference in its entirety. Methods for preparing microparticulate β-glucan particles are disclosed in U.S. Pat. No. 5,702,719, the disclosure of which is incorporated herein by reference in its entirety. Microparticulate glucan product can also be obtained with the average particle size of about 1.0 microns or less or about 0.20 microns or less.

β-glucan particles can be reduced in size by mechanical means such as by, using a blender, microfluidizer, or ball mill, for example. For example, particle size can be reduced using a blender having blunt blades, wherein the glucan mixture is blended for a sufficient amount of time, preferably several minutes, to completely grind the particles to the desired size without overheating the mixture. Another grinding method comprises grinding the glucan mixture in a ball mill with 10 mm stainless steel grinding balls. This latter grinding method is particularly preferred when a particle size of about 0.20 microns or less is desired.

Prior to grinding, the glucan mixture is preferably passed through a series of sieves, each successive sieve having a smaller mesh size than the former, with the final mesh size being about 80. The purpose of sieving the mixture is to separate the much larger and more course glucan particles from smaller particles (the pore size of an 80 mesh sieve is about 0.007 inches or 0.178 mm). The separated larger particles are then ground down as described above and re-sieved to a final mesh size of 80. The process of sieving and grinding is repeated until a final mesh size of 80 is obtained. The sieved particles are combined and ground down further, preferably for at least an hour, until the desired particle size is obtained, preferably about 1.0 micron or less, more preferably about 0.20 microns or less. Periodic samples of the fine grind glucan are taken during the grinding process and measured using a micrometer on a microscope.

### Pharmaceutical Formulations

### Administration

The administration of the whole glucan particles and agent can be administered sequentially, co-administered or in multiple dosing. Further, the order of administration is interchangeable.

### Formulation

Oral formulations suitable for use in the practice of the present invention include capsules, gels, cachets, tablets, effervescent or non-effervescent powders or tablets, powders or granules; as a solution or suspension in aqueous or non-aqueous liquid; or as an oil-in-water liquid emulsion or a water-in-oil emulsion. Or in a formulation for delivery intranasally. The compounds of the present invention may also be presented as a bolus, electuary, or paste.

Generally, formulations are prepared by uniformly mixing the active ingredient with liquid carriers or finely divided solid carriers or both, and then if necessary shaping the product. A pharmaceutical carrier is selected on the basis of the chosen route of administration and standard pharmaceutical practice. Each carrier must be “acceptable” in the sense of being compatible with the other ingredients of the formulation and not injurious to the subject. This carrier can be a solid or liquid and the type is generally chosen based on the type of administration being used. Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g., PEG, are also preferred carriers.

The formulations for oral administration may comprise a non-toxic, pharmaceutically acceptable, inert carrier such as lactose, starch, sucrose, glucose, methyl cellulose, magnesium stearate, dicalcium phosphate, calcium sulfate, mannitol, sorbitol, cyclodextrin, cyclodextrin derivatives, or the like.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc. Nebulized formulation for inhalation can include sodium chloride, sodium saccharine or sorbitani trioleas, whereas inhalation via compressed carbonated formulation in a puffer can include 1,1,1,2-tetrafluoroethanum, monofluorotrichloromethanum tetrafluorodichloroaethanum or difluorodichloromethanum.

Capsule or tablets can be easily formulated and can be made easy to swallow or chew. Tablets may contain suitable carriers, binders, lubricants, diluents, disintegrating agents, coloring agents, flavoring agents, flow-inducing agents, or melting agents. A tablet may be made by compression or molding, optionally with one or more additional ingredients. Compressed tables may be prepared by compressing the active ingredient in a free flowing form (e.g., powder, granules) optionally mixed with a binder (e.g., gelatin, hydroxypropylmethylcellulose), lubricant, inert diluent, preservative, disintegrant (e.g., sodium starch glycolate, cross-linked carboxymethyl cellulose) surface-active or dispersing agent. Suitable binders include starch, gelatin, natural sugars such as glucose or β-lactose, corn sweeteners, natural and synthetic gums such as acacia, tragacanth, or sodium alginate, carboxymethylcellulose, polyethylene glycol, waxes, or the like. Lubricants used in these dosage forms include sodium oleate, sodium stearate, magnesium stearate, sodium benzoate, sodium acetate, sodium chloride, or the like. Disintegrators include, for example, starch, methyl cellulose, agar, bentonite, xanthan gum, or the like. Molded tablets may be made by molding in a suitable machine a mixture of the powdered active ingredient moistened with an inert liquid diluent.

The tablets may optionally be coated or scored and may be formulated so as to provide slow- or controlled-release of the active ingredient. Tablets may also optionally be provided with an enteric coating to provide release in parts of the gut other than the stomach.

Exemplary pharmaceutically acceptable carriers and excipients that may be used to formulate oral dosage forms of the present invention are described in U.S. Pat. No. 3,903,297 to Robert, issued Sep. 2, 1975, incorporated by reference herein in its entirety. Techniques and compositions for making dosage forms useful in the present invention are described in the following references: 7 Modem Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976). In certain embodiments, the dosing of glucan administered is selected from the range of about 0 to about 6,000 mg/day or from about 0 to about 100 mg/kg/day. For example, the dosage can be about 66.6 μg/day or about 200 μg/day or about 666.6 μg/day.

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Extemporaneous injections solutions and suspensions may be prepared from sterile powders, granules and tablets of the kind previously described. Parenteral and intravenous forms may also include minerals and other materials to make them compatible with the type of injection or delivery system chosen.

All publications cited are incorporated by reference in their entirety. The present invention is now illustrated by the following Exemplification, which is not intended to be limiting in any way.

### EXEMPLIFICATION

### Example 1

### Evaluation of the Protective Effect of Immune Modulators Using an Experimental Murine Influenza Model

### Experimental Conditions

32 Balb/c mice divided in 4 groups of 8 animals each.

Group 1: Negative control (gavaged H₂O)
Group 2: Imucell WGP glucan (Biopolymer Engineering, 20 mg/kg in 100 ml of H₂O)
Group 3: Negative control (untreated).

Mice in groups 1 and 2 received the respective treatment per os (Gavage with needle B-D #20) during 8 consecutive days.

One or 2 hours after the last gavage, mice from all the four groups were anaesthetized then infected intra-nasally with 10 LD₅₀ (10^{2.56}TCID₅₀) of human influenza virus A/PR/8/34 adapted to grow in mice in our labs.

Each experiment group was divided into 2 subgroups in order to do the viral load in the lungs:
Subgroup 1: 4 animals/group (50% of the animals) were sacrificed at Day 5 p.i. in order to evaluate the viral load in the lungs using two different techniques (HAU and TCID₅₀).
Subgroup 2: 4 animals per group (50% of the animals) were followed in order to evaluate survival at Day 14.

The body weight of each animal was taken regularly (every 2 to 3 days):
Before starting the gavages (Gavage),
Day 0 (Infection)
Day 3 p.i.
Day 5 p.i.
Day 7 p.i.
All animals (except one) died at Day 9 p.i.
   Results are presented in 4 tables
(1) Table 1: Body Weight and calculation of changes in body weight (% weight loss) post infection (Subgroup 1: Animals sacrificed at Day 5 p.i. for evaluation of viral load in the lungs)
(2) Table 2: Viral load calculated by two techniques (HAU and TCID₅₀).
(3) Table 3: Body Weight and calculation of changes in body weight (% weight loss) post infection (Subgroup 2)
(4) Table 4: Survival at Day 14 p.i. (Subgroup 2)

TABLE 1
Body Weight and calculation of changes in body weight (% weight loss)
post infection (Subgroup 1: Animals sacrificed at Day 5 p.i.)

| | Body Weight (in grams) | | | | % of weight loss | | |
|---|---|---|---|---|---|---|---|
| Treatment | | | Day 3 | Day 5 | | Day 3 | Day 5 |
| (Mouse ID#) | Gavage | Infection | p.i. | p.i. | Gavage | p.i. | p.i. |
| H₂O (#1) | 15.58 | 16.18 | 14.85 | 12.05 | NA | 8.2 | 25.5 |
| H₂O (#2) | 15.21 | 16.28 | 13.52 | 13.62 | NA | 16.9 | 16.3 |
| H₂O (#3) | 15.75 | 16.34 | 14.85 | 14.11 | NA | 9.1 | 13.6 |
| H₂O (#5) | 16.18 | 17.10 | 15.27 | 14.63 | NA | 10.7 | 14.4 |
| Mean | 15.68 | 16.48 | 14.62 | 13.60 | NA | 11.23 | 17.45 |
| SD | 0.40 | 0.42 | 0.76 | 1.11 | | 3.92 | 548 |
| WGP glucan (#17) | 16.54 | 17.01 | 15.18 | 13.17 | NA | 10.7 | 225 |
| WGP glucan (#18) | 16.96 | 17.31 | 15.28 | 15.11 | NA | 11.7 | 12.7 |
| WGP glucan (#19) | 16.88 | 17.18 | 15.93 | 13.09 | NA | 7.2 | 23.8 |
| WGP glucan (#20) | 17.47 | 17.70 | 15.95 | 13.77 | NA | 9.8 | 22.2 |
| Mean | 1696 | 17.30 | 15.59 | 13.79 | NA | 9.85 | 20.3 |
| SD | 0.38 | 0.29 | 0.41 | 0.93 | | 1.93 | 5.11 |
| Untreated (#25) | 16.42 | 17.32 | 15.90 | 14.23 | NA | 8.1 | 17.8 |
| Untreated (#26) | 15.99 | 15.80 | 14.72 | 12.25 | NA | 6.8 | 22.4 |
| Untreated (#27) | 14.85 | 14.73 | 14.77 | 12.50 | NA | 5.5 | 20.0 |
| Untreated (#28) | 13.80 | 16.16 | 13.48 | 11.33 | NA | 8.4 | 23.0 |
| Mean | 15.27 | 16.0 | 14.72 | 12.58 | NA | 7.2 | 20.8 |
| SD | 1.18 | 1.07 | 0.99 | 1.21 | | 1.33 | 235 |

NA: non applicable (i.e., animals did not loose any weight)

### CONCLUSION

No significant change in body weight loss was found in all 4 groups of animals. In both negative groups (Groups 1 and 4) no significant change in body weight could be observed.

This observation is the same as previously obtained with this experimental animal model and as expected of animals infected with an 10 LD₅₀ and sacrificed at Day 5 post-infection.

Furthermore, there was no change in body weight loss in both experimental groups (Groups 2 and 3)

TABLE 2
Viral titres in the lungs (Day 5 p.i. (Subgroup 1)

| Treatment | | |
|---|---|---|
| (Mouse ID #) | HAU/ml | TCID₅₀/ml |
| H₂O (#1) | 20 | 10^{3.4} |
| H₂O (#2) | 20 | 10^{3.5} |
| H₂O (#3) | 120 | 104.5 |
| H₂O (#5) | 80 | 105 |
| Mean | 60 | 10^{4.1} |
| SD | 49 | |
| WGP glucan (#17) | 160 | 10^{4.75} |
| WGP glucan (#18) | 20 | 10^{4.5} |
| WGP glucan (#19) | 20 | 10^{3.5} |
| WGP glucan (#20) | 60 | 10^{4.75} |
| Mean | 65 | 10^{4.4} |
| SD | 66 | |
| Untreated (#25) | 80 | 10^{4.2} |
| Untreated (#26) | 80 | 10^{4.6} |
| Untreated (#27) | 80 | 10^{5.2} |
| Untreated (#28) | 40 | 10^{4.4} |
| Mean | 70 | 10^{4.6} |
| SD | 20 | |

### Conclusion

No reduction in the viral load in the experimental animal groups (Group 2) as compared to the negative controls (Group 1 and 3).

This is, however, a small experiment, and the number of animal used was very limited. Moreover, both techniques show large individual variability.

TABLE 3
Body Weight and calculation of changes in body weight (% weight loss) post infection (Subgroup 2)

| | Body weigh (in grams) | | | | | | | | |
|---|---|---|---|---|---|---|---|---|---|
| Treatment | | | Day 3 | Day 5 | Day 7 | % of Body weight loss | | | |
| (Mouse ID #) | Gavage | Infection | p.i. | p.i. | p.i. | Gavage | Day 3 | Day 5 | Day 7 |
| H₂O (#4) | 18.38 | 18.19 | Dead | | | NA | | | |
| H₂O (#6) | 18.07 | 18.64 | 17.63 | 15.30 | 13.58 | NA | 5.4 | 17.9 | 27.15 |
| H₂O (#7) | 17.25 | 17.35 | 15.60 | 12.94 | 12.09 | NA | 10.1 | 25.4 | 3032 |
| H₂O (#8) | 14.16 | 14.87 | 13.45 | 11.58 | 10.00 | NA | 9.5 | 22.1 | 32.75 |
| Mean | 16.97 | 17.26 | 12.23 | 13.27 | 11.89 | NA | 8.33 | 21.80 | 30.07 |
| WGP glucan (#21) | 14.90 | 15.15 | 16.50 | 11.50 | 10.17 | NA | 8.9 | 24.0 | 32.87 |
| WGP glucan (#22) | 15.59 | 16.39 | 15.05 | 12.95 | Dead | NA | 8.1 | 20.9 | — |
| WGP glucan (#23) | 16.08 | 17.84 | 13.25 | 14.88 | 12.78 | NA | 25.7 | 16.5 | 28.36 |
| WGP glucan (#24) | 16.02 | 16.89 | 15.70 | 14.72 | 13.17 | NA | 7.0 | 12.8 | 22.02 |
| Mean | 15.65 | 16.57 | 15.13 | 13.51 | 12.04 | NA | 12.43 | 18.55 | 27.75 |
| SD | 0.54 | 1.12 | 1.38 | 1.60 | 1.63 | | 8.88 | 4.92 | 5.45 |
| Untreated (#29) | 15.02 | 16.16 | 14.47 | 12.75 | 11.28 | NA | 10.4 | 20.8 | 30.20 |
| Untreated (#30) | 17.12 | 18.31 | 16.34 | 14.05 | 12.27 | NA | 10.7 | 23.2 | 32.99 |
| Untreated (#31) | 16.27 | 17.70 | 15.90 | 13.53 | 11.95 | NA | 10.1 | 23.5 | 32.49 |
| Untreated (#32) | 15.51 | 16.30 | 14.79 | 13.10 | 11.67 | NA | 9.8 | 16.9 | 28.40 |
| Mean | 15.98 | 17.12 | 15.38 | 13.36 | 11.79 | NA | 10.25 | 21.10 | 31.02 |
| SD | 0.92 | 1.06 | 0.89 | 0.56 | 0.42 | | 0.39 | 3.05 | 2.13 |

* NA = non applicable (i.e., animals did not loose any weight)

### Conclusion

Body weight loss could be calculated until Day 7 mainly because very few animals survived the infection after Day 9 (see Table 4).

When compared to Group 3 (untreated negative controls) animals in group 2 (WGP glucan) seem to have lost less body weight that in the controls. This is not however, significant because of the small number of animals in this experiment.

TABLE 4
Survival of animals infected with 10 LD₅₀ (Subgroup 2)

| | Group/Mice ID | | | | | |
|---|---|---|---|---|---|---|
| | Day 7 | Day 9 | Day 11 | Day 12 | Day 13 | Day 14 |
| | p.i. | p.i. | p.i. | p.i. | p.i. | p.i. |
| H₂O (#1) | 3/4 | 0/4 | 0/4 | 0/4 | 0/4 | 0/4 |
| WGP glucan | 3/4 | 2/4 | 2/4 | 1/4 | 1/4 | 37989 |
| (#3) | | | | | | |
| Untreated | 4/4 | 0/4 | 0/4 | 0/4 | 0/4 | 37989 |
| (#4) | | | | | | |

### Conclusion

The only group where we can see a tendency for a protection is the WGP glucan treated group (Group 3). In this group 50% of the animal survived beyond Day 9 when all the animals from all groups died.
One animal died on Day 12 p.i. this animal had lost 38,28% of its weight on Day 11.

This group of animals was observed until Day 14 p.i. and only one animal survived, this animal had however lost 35,17% of its body weight at Day 11 and 40.67% at Day 14 p.i., but otherwise looked healthy.

In summary, a tendency for protection only using WGP glucan was observed as compared to the negative control.

### Example 2

### Materials and Methods

### Mean Survival Time & Total Survival Model

BALB/C females, 6-8 weeks old on arrival
Bacillus anthracis Vollum 1B, infected s.c.
Challenge dose from 1 LD₅₀ to 10 LD₅₀

Immune modulators given either orally or s.c depending on the drug and experiment. Single dose administration Day-2, Multiple dose administration Day-7 to 0.

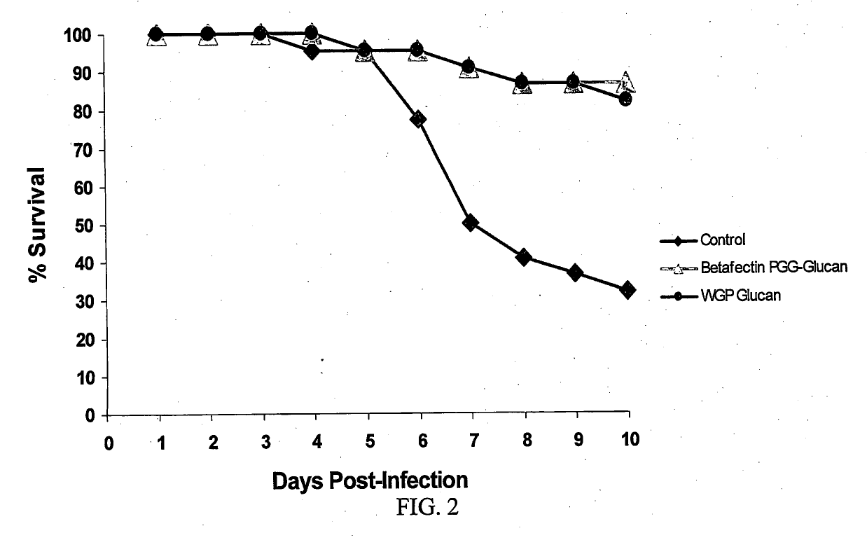

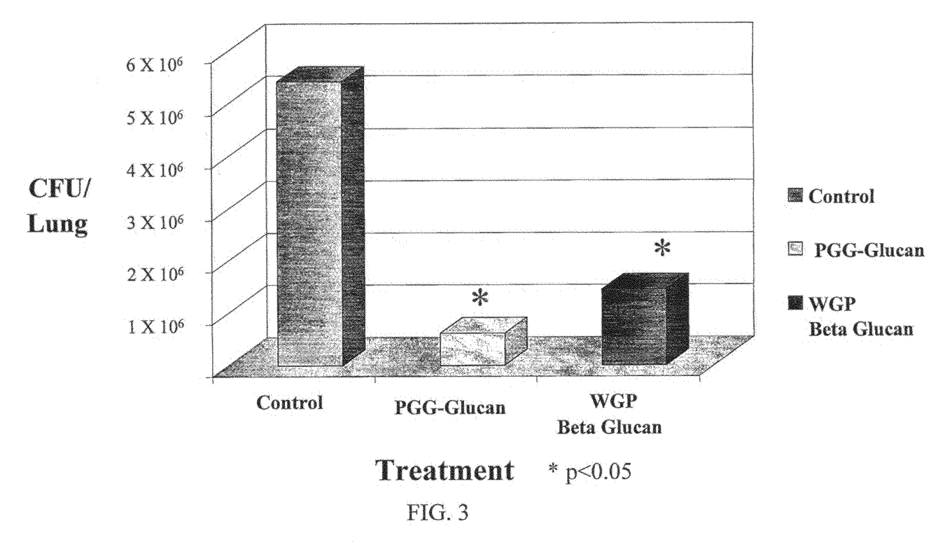

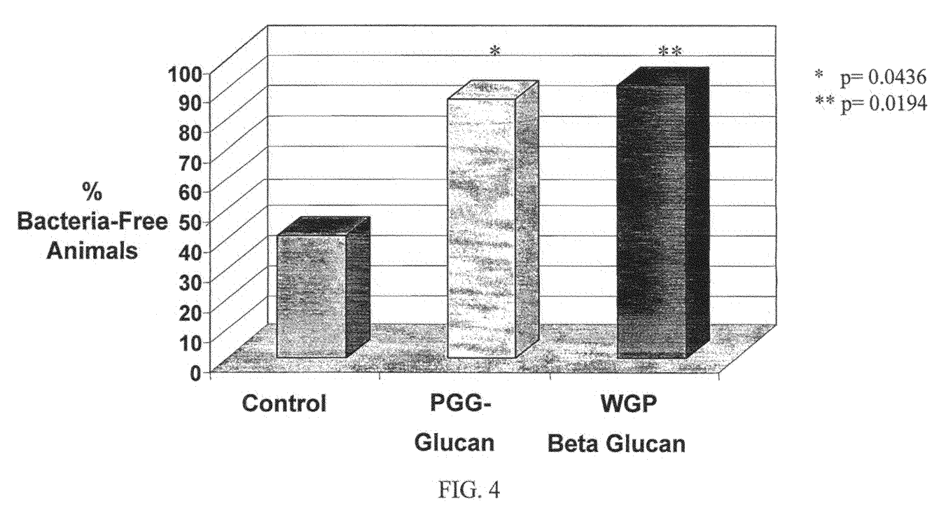

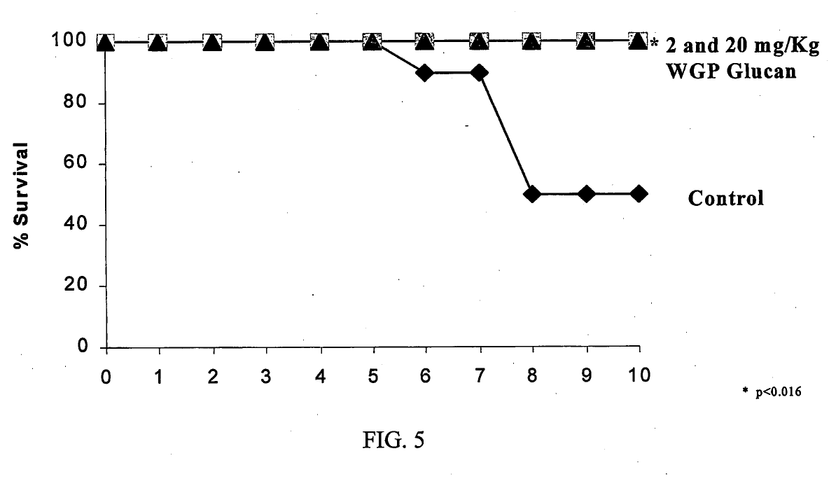

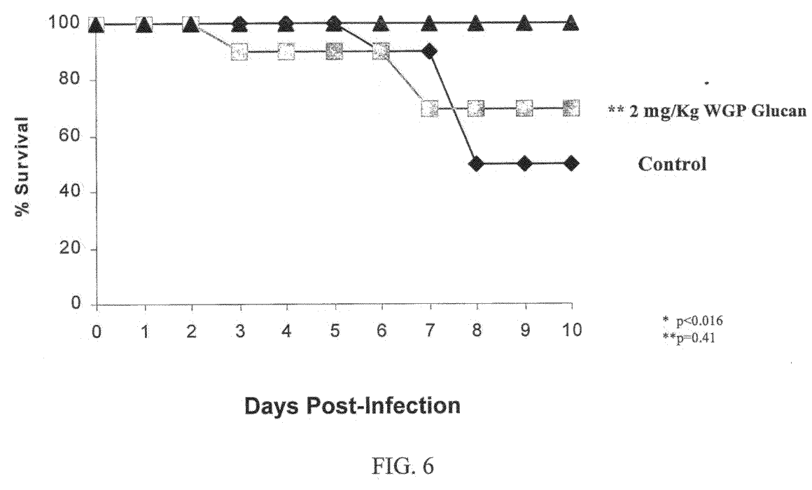

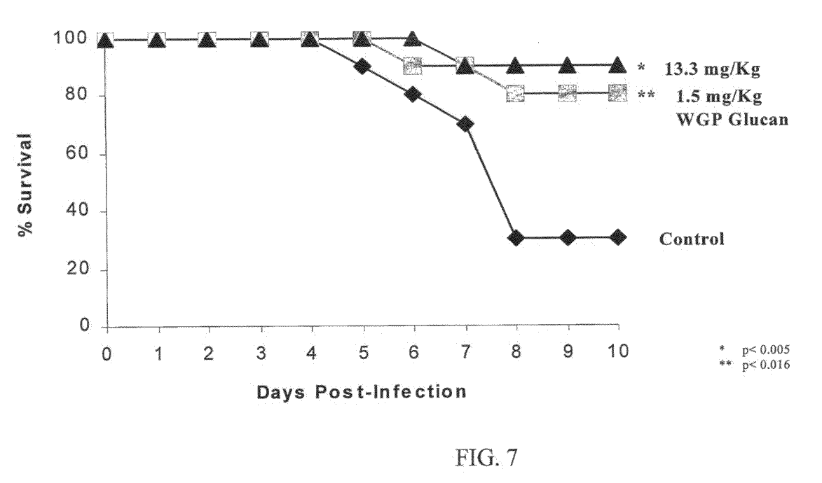

Experiments ran 10 days post-challenge.
FIGS. 2-7 show the data from this experiment

TABLE 5

| | Alone | Cipro | Vaccine 1X | Vaccine 2X |
|---|---|---|---|---|
| Control | 4.3 | 4.8 | 5.6 | 5.0 |
| | 0/10 | 0/8 | 1/8 | 1/8 |
| WGP | 5.6 | [symbol] | 7.0 | 7.6 |
| 66.6 μg | 0/10 | 1/10 | 2/10 | 2/10 |
| WGP | 6.8 | 8.3 | 8.1 | 8.5 |
| 200 μg | 2/10 | 3/10 | 3/8 | 4/8 |
| WGP | 7.6 | 6.4 | 8.3 | 9.0 |
| 666.7 μg | 3/10 | 0/10 | 4/8 | 5/8 |

Mean Survival Time x.y
Total Survival x/y
Bold P ≦ 0.05

### Mean Survival Time x.y Total Survival x/y Bold P≦0.05

TABLE 6
Enhanced Survival Time-Ciprofloxacin

| | No Treatment | Ciprofloxacin |
|---|---|---|
| No Treatment | 4.3 | 4.8 |
| 66.6 μg | 5.6 | 7.1 |
| BEI-O-201 | | |

The challenge dose was 10LD₅₀ of Bacillus anthracis. 66.6 μg WGP (BEI-O-201, Biopolymer Engineering Inc., Eagan, Minn.) was administered eight times on a daily basis beginning 7 days before anthrax challenge. 250 μg Liposome encapsulated Ciprofloxacin was administered in a 0.1 mL volume s.c one day prior to challenge. The italics data is statistically different than controls (p<0.05) by t-test.

TABLE 7

| | No treatment | Vaccine 1X | Vaccine 2X |
|---|---|---|---|
| No treatment | 0/10 | 1/8 | 1/8 |
| 66.6 μg | 38361 | 2/8 | 2/8 |
| WGP | | | |
| 200 μg | 2/10 | 3/8 | 4/8 |
| WGP | | | |
| 666.6 μg | 3/10 | 4/8 | 5/8 |
| WGP | | | |

Table 7 is a chair showing the challenge dose was 10 LD₅₀ of Bacillus anthracis. WGP was administered eight times on a daily basis beginning 7 days before anthrax challenge. Vaccine was given on Day 7 alone (Vaccine 1x) or Day 7 and Day 1 (Vaccine 2x) prior to anthrax challenge. The results in italics are statistically different than no treatment controls (p<0.05) by Fischer Exact test.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating influenza comprising orally administering whole glucan particles.

2. A method of treating rhinovirus, comprising orally administering whole glucan particles.

3. A method of treating bacterial infections, comprising orally administering whole glucan particles and an antibiotic directed to the bacterial infection.

4. The method of claim 3, wherein the antibiotic is ciprofloxacin.

5. The method of treating viral or bacterial infection comprising orally administering whole glucan particles and a vaccine or complement activating monoclonal antibody or complement activating polyclonal antibody.

6. A method of enhancing glucan-mediated immunogenic response via the complement system, comprising administering to an individual a therapeutically effective orally bioavailable amount of whole glucan particles and agent, wherein the agent activates the complement system and the glucan enhances immunogenic response whereby enhancing the activity of the agent.

7. The method of claim 6, wherein the agent is a viral or bacterial monoclonal antibody or vaccine.

8. The method of claim 6, wherein the orally administered glucan is taken up by macrophages, transported to the bone marrow and other immune organs, degraded and the released fragments activate the immunogenic cells.

9. A method of treating or preventing pathogenesis of viral infection in humans or animals by one or more infectious agents comprising administering a prophylactically or therapeutically effective amount of whole glucan particles and an antiviral agent to the human or animal, wherein the agent activates the complement system and the glucan enhances immunogenic response, by enhancing the activity of the agent.

10. A method of treating or preventing pathogenesis of bacterial infection in humans or animals by one or more infectious agents comprising administering a prophylactically or therapeutically effective amount of whole glucan particles and an antibiotic to the human or animal, wherein the agent activates the complement system and the glucan enhances immunogenic response, by enhancing the activity of the agent.

11. A method of treating or preventing pathogenesis of an infection in humans or animals by one or more infectious agents comprising administering to the human or animal a prophylactically or therapeutically effective amount of whole glucan particles and a vaccine to the infectious agent, wherein the vaccine activates the complement system and the glucan enhances immunogenic response, by enhancing the activity of the vaccine.

12. The method of claim 1, wherein the glucan is administered in a dose range of about 0 to about 6000 mg per day or about 0 to about 100 mg/kg/day.
